SEQUENCE LISTING (7 sequences)

SEQ ID NO: 1 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - DNA, 24 bp, Artificial Sequence
  Primer
ggtaccatgccgccaccccggacc

SEQ ID NO: 3 - DNA, 26 bp, Artificial Sequence
  Primer
tctagaggcatcaacaactggaaaag

SEQ ID NO: 4 - DNA, 27 bp, Artificial Sequence
  Primer
cagcaagcagtgaccgccccgagccgc

SEQ ID NO: 5 - DNA, 27 bp, Artificial Sequence
  Primer
gcggctcggggcggtcactgcttgctg

SEQ ID NO: 6 - DNA, 27 bp, Artificial Sequence
  Primer
tgcgccccgagccccgtttctttcctc

SEQ ID NO: 7 - DNA, 27 bp, Artificial Sequence
  Primer
gaggaaagaaacggggctcggggcgca

(19) United States
(12) Patent Application Publication
(10) Pub. No.: US 2014/0351964 A1
(43) Pub. Date: Nov. 27, 2014

(54) TRANSGENIC MOUSE MODEL FOR DEVELOPING ENZYME REPLACEMENT THERAPY FOR IDURONATE-2-SULFATASE DEFICIENCY SYNDROME

(71) Applicant: MEDIGENEBIO CORPORATION, GYEONGGI-DO (KR)

(72) Inventor: Thong-Gyu Jin, Seoul (KR)

(73) Assignee: MEDIGENEBIO CORPORATION, GYEONGGI-DO (KR)

(21) Appl. No.: 14/294,681

(22) Filed: Jun. 3, 2014

Related U.S. Application Data
(63) Continuation of application No. 12/986,852, filed on Jan. 7, 2011, now abandoned.

(51) Int. Cl. A01K 67/027

(52) U.S. Cl. CPC A01K 67/0276; A01K 2267/03; A01K 2227/105
USPC 800/9

(57) ABSTRACT

The present invention relates to a transgenic mouse model for developing enzyme replacement therapy for iduronate-2-sulfatase deficiency syndrome, for example, Hunter syndrome. More specifically, the present invention relates to a transgenic mouse to be used for developing enzyme replacement therapy for iduronate-2-sulfatase, wherein the immune response against injected enzyme, such as, recombinant iduronate-2-sulfatase has been minimized in transgenic mouse model in the course of treating in vivo iduronate-2-sulfatase replacement.

### CROSS-REFERENCE TO RELATED APPLICATIONS

This application is a continuation of U.S. patent application Ser. No. 12/986,852, filed Jan. 7, 2011. The entire contents of the aforementioned patent application is incorporated herein by reference.

### SEQUENCE LISTING

The instant application contains a Sequence Listing which has been submitted in ASCII format via EFS-Web and is hereby incorporated by reference in its entirety. Said ASCII copy, created on Jun. 3, 2014, is named 90939CON310754_ST25.txt and is 6,162 bytes in size.

### BACKGROUND OF THE INVENTION

1. Field of the Invention

The present invention relates to a transgenic mouse model for developing enzyme replacement therapy for iduronate-2-sulfatase deficiency syndrome, for example, Hunter syndrome. More specifically, the present invention relates to a transgenic mouse to be used for developing enzyme replacement therapy for iduronate-2-sulfatase, wherein the immune response against injected enzyme, such as, recombinant iduronate-2-sulfatase has been minimized in the transgenic mouse model in the course of treating in vivo iduronate-2-sulfatase replacement.

2. Description of Prior Art

Mucopolysaccharidosis is an inherited metabolic disorder caused by the absence or malfunction of lysosomal enzyme required for degenerating the glycosaminoglycan, one of long chain of sugar carbohydrates in the cell, which helps to build the bone, cartilage, tendon, cornea, skin and connective tissue.

Mucopolysaccharidosis (MPS) type II has been called Hunter disease resulted from absence or insufficient levels of iduronate-2-sulfatase (EC 3.1.6.13). Iduronate-2-sulfatase is an enzyme involved in the degeneration of glycosaminoglycans, such as, dermatan sulfate and heparan sulfate, which are found in the lysosome in the cell. The deficiency of iduronate-2-sulfatase results in the accumulation of heparin and dermatan sulfate within phagocytes, endothelium, smooth muscle cells, neurons, and fibroblasts.

The symptoms of Hunter syndrome are generally not apparent at birth, but usually start to become noticeable after the first year of life. Often, the first symptoms of Hunter syndrome may include abdominal hernias, ear infections, runny noses, and colds. Since these symptoms are quite common among all infants, they are not likely to lead a doctor to make a diagnosis of Hunter syndrome right away. As the buildup of glycosaminoglycan continues throughout the cells of the body, signs of Hunter syndrome become more visible. Physical appearances of many children with Hunter syndrome include a distinctive coarseness in their facial features, including a prominent forehead, a nose with a flattened bridge and an enlarged tongue. For this reason, unrelated children with Hunter syndrome often look alike. They may also have a large head as well as an enlarged abdomen. Many continue to have frequent infections of the ears and respiratory tract.

To develop the enzyme for treating Hunter syndrome, a knock-out mouse model for developing the agent has been designed. In Korean Patent No. 10-884,564, a knock-out mouse which is homozygous for a disruption in the iduronate-2-sulfatase (IDS) gene has been disclosed. In this disclosure, it has been also disclosed that a knock-out mouse can be used for evaluating therapeutic enzyme in treating mucopolysaccharidosis Type II, by administering the therapeutic enzyme to the mouse, and evaluating the mouse for tissue pathology associated with iduronate-2-sulfatase deficiency.

However, it is doubtful if a knock-out mouse is the best model for evaluating the ability of targeting a system to deliver a therapeutic enzyme to selected tissues or organs by administering an effective amount of iduronate-2-sulfatase, because the knock-out mouse has a handicap of generating antibody as to recombinant iduronate-2-sulfatase at the time of carrying out enzyme replacement therapy.

For treating a mucopolysaccharidosis (MPS) type II, Shire Human Genetic Therapies Ltd (Lexington, Mass., USA) has developed the enzyme of iduronate-2-sulfatase as trade name of Elaprase® by recombinant DNA technology in a human cell line. In 2006, this enzyme drug has been approved for treating Hunter syndrome by the FDA. Recently, this drug has been commercially marketed in about 15 countries.

Direct enzyme replacement therapy has been carried out for administering an Elaprase® or its analogue for treating Hunter syndrome. However, this enzyme replacement therapy of Elaprase® has an adverse effect of generating anti-Elaprase® IgG antibodies in a patient's serum when Elaprase® has been administered more than 53 weeks.

Therefore, other therapies, for example, cell implant or gene therapy using a vector also have been tried for treating Hunter syndrome. However, any of the therapies developed until now cannot show a sufficient treatment effect. Further, we cannot estimate which treatment method will be effective for an individual patient.

On the other hand, a transgenic mouse tolerant to human N-acetylgalactosamine-6-sulfate sulfatase has been developed and disclosed in Human Molecular Genetics, 2005, Vol. 14, No. 22, pp. 3321.about.3335. The deficiency of N-acetylgalactosamine-6-sulfate sulfatase (GALNS) (EC 3.1.6.4) causes mucopolysaccharidosis (MPS) type VIA. Therefore, this transgenic mouse can be considered as an optimal animal model for treating mucopolysaccharidosis (MPS) type VIA syndrome for the following reasons.

This mouse model has substantial storage of un-degraded glycosaminoglycans in bones and hepatocytes, because it has biochemical properties more similar to human MPS IVA than the original MPS IVA knock-out mouse. The mouse lacks GALNS activity because the Gains gene (which corresponds to the active site mutation C79S in the human GALNS gene) and confers tolerance to human GALNS by targeting human inactive GALNS cDNA with C79S mutation in the mouse Gains gene. This mouse model can be regarded as a useful tool for evaluating enzyme replacement therapy as to the MPS WA patients.

An ordinary expert in the field of transgenic mouse may consider that the best animal model for developing enzyme replacement therapy for iduronate-2-sulfatase as well as Elaprase® can be established if a transgenic mouse tolerant to human iduronate-2-sulfatase can be actualized.

However, a transgenic mouse which is produced using a targeting vector, where site directed mutagenesis of C84T (cysteine, the 84ₜₕ amino acid of human iduronate-2-sulfatase is replaced by threonine) or R88P (arginine, 88^{th} amino acid of human iduronate-2-sulfatase is replaced by proline) has been made in a human iduronate-2-sulfatase gene cannot be adopted as an optimal animal model because of the following handicaps.

Such a transgenic mouse can express phenotype inactive iduronate-2-sulfatase, which does not cause the formation of anti-IDS IgG antibody. However, some neutralized antibody is generated in the course of IDS enzyme injection to said transgenic mouse, which results in adverse sensitization to the injected IDS enzyme. Therefore, said transgenic mouse cannot be used as an animal model for developing enzyme replacement therapy for iduronate-2-sulfatase.

To solve the above problems, the inventor of the present application has developed a new transgenic mouse model tolerant to iduronate-2-sulfatase by cross-breeding between said transgenic mouse C84T or R88P and iduronate-2-sulfatase knock-out mouse.

### SUMMARY OF THE INVENTION

The object of present invention is to provide a transgenic mouse tolerant to human iduronate-2-sulfatase without generating anti-IDS IgG antibody prepared by the steps comprising

i) preparing an iduronate-2-sulfatase knock-out mouse comprising disruption in exon 2 and exon 3 in the iduronate-2-sulfatase gene, wherein said disruption has been introduced into its genome by homologous recombination with DNA targeting construct in an embryonic stem cell such that the targeting construct is stably integrated in the genome of said mouse, wherein the disruption of the iduronate-2-sulfatase gene results in an inability of said mouse to produce detectable levels of iduronate-2-sulfatase;

ii) preparing an iduronate-2-sulfatase mutation mouse comprising site directed mutagenesis of iduronate-2-sulfatase gene, wherein cysteine, the 84^{th} amino acid of human iduronate-2-sulfatase of SEQ ID No:1 is replaced by threonine (C84T) or arginine, the 88^{th} amino acid of human iduronate-2-sulfatase of SEQ ID No:1 is replaced by proline (R88P), wherein said mutation has been introduced into its genome with DNA targeting construct in an embryonic stem cell such that the targeting construct is stably integrated in the genome of said mouse, wherein the mutation of the iduronate-2-sulfatase gene results in the capability of said mouse to produce inactive type of iduronate-2-sulfatase; and

iii) cross-breeding the knock-out mouse prepared in step (i) and the mutation mouse prepared in step (ii) for obtaining the transgenic mouse tolerant to iduronate-2-sulfatase.

Further, in the iduronate-2-sulfatase knock-out mouse, said DNA targeting construct comprises 2027 by of left arm, 1800 by of neomycin resistance gene (PGK-neo) and 5393 by of right arm, wherein the left arm is constructed from residue 12133 to residue 14160 of the wild type allele of human iduronate-2-sulfatase gene, the right arm is constructed from residue 15643 to residue 21036 of the wild type allele of human iduronate-2-sulfatase gene, and said neomycin resistance gene (PGK-neo) is flanked by LoxP sites where residue 14161 to residue 15642 of the wild type allele of human iduronate-2-sulfatase gene are deleted.

Further, in the iduronate-2-sulfatase mutation mouse (C84T), mutated cDNA in the DNA targeting construct has been prepared with the following steps;

i) preparing a human iduronate-2-sulfatase cDNA by amplifying wild type allele using forward primer of SEQ ID No:2 and reverse primer of SEQ ID No:3; and

ii) preparing a mutated human iduronate-2-sulfatase cDNA by amplifying the cDNA prepared in step (i) subjected to site directed mutagenesis using primer of SEQ ID No:4 and reverse primer of SEQ ID No:5.

Further, in the iduronate-2-sulfatase mutation mouse (R88P), mutated cDNA in the DNA targeting construct has been prepared with the following steps;

i) preparing a human iduronate-2-sulfatase cDNA by amplifying wild type allele using forward primer of SEQ ID No:2 and reverse primer of SEQ ID No:3; and

ii) preparing a mutated human iduronate-2-sulfatase cDNA by amplifying the cDNA prepared in step (i) subjected to site directed mutagenesis using primer of SEQ ID No:6 and reverse primer of SEQ ID No:7.

A further object of the present invention is to provide a transgenic mouse model for developing enzyme replacement therapy for iduronate-2-sulfatase deficiency syndrome, wherein the target enzyme for treating iduronate-2-sulfatase deficiency syndrome has been experimented and selected by the following steps;

i) preparing a transgenic mouse tolerant to human iduronate-2-sulfatase without generating anti-IDS IgG antibody;

ii) administering a candidate enzyme to said transgenic mouse;

iii) measuring the efficacy of the candidate enzyme and adverse effect caused by anti-IDS IgG antibody; and

iv) selecting the optimal enzyme showing maximum efficacy and minimum adverse effect among candidate enzymes.

### BRIEF DESCRIPTION OF THE DRAWINGS

FIG. 1 shows the construction of a targeting vector for preparing an iduronate-2-sulfatase knock-out mouse. Said targeting vector consists of left arm of 2027 bp, neomycin resistance gene (PGK-neo) of 1800 by and right arm of 5393 bp. The left arm is constructed from residue 12133 to residue 14160 of the wild type allele of iduronate-2-sulfatase gene. The right arm is constructed from residue 15643 to residue 21036 of the wild type allele of iduronate-2-sulfatase gene. The neomycin resistance gene (PGK-neo) is flanked by LoxP sites where residue 14161 to residue 15642 of the iduronate-2-sulfatase gene are deleted.

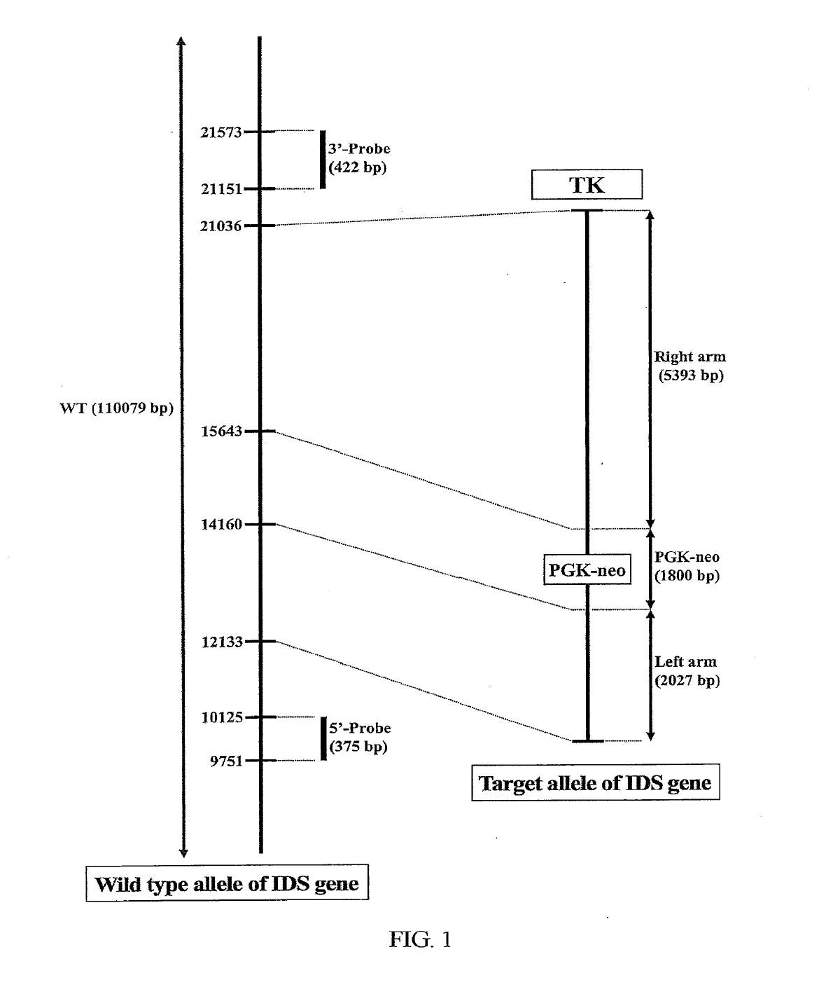

FIG. 2 shows the construction of targeting vector for preparing an iduronate-2-sulfatase knock-out mouse. The neomycin resistance gene (PGK-neo) is flanked by LoxP sites where part of iduronate-2-sulfatase gene from residue 14161 to residue 15642 is deleted. The wild type allele of the iduronate-2-sulfatase gene is digested by Xho1 at residue 14160 and the wild type allele of the iduronate-2-sulfatase gene is digested by EcoR1 at residue 15643. Then, the neomycin resistance gene (PGK-neo) is flanked by LoxP sites where residue 14161 to residue 15642 of the iduronate-2-sulfatase gene are deleted.

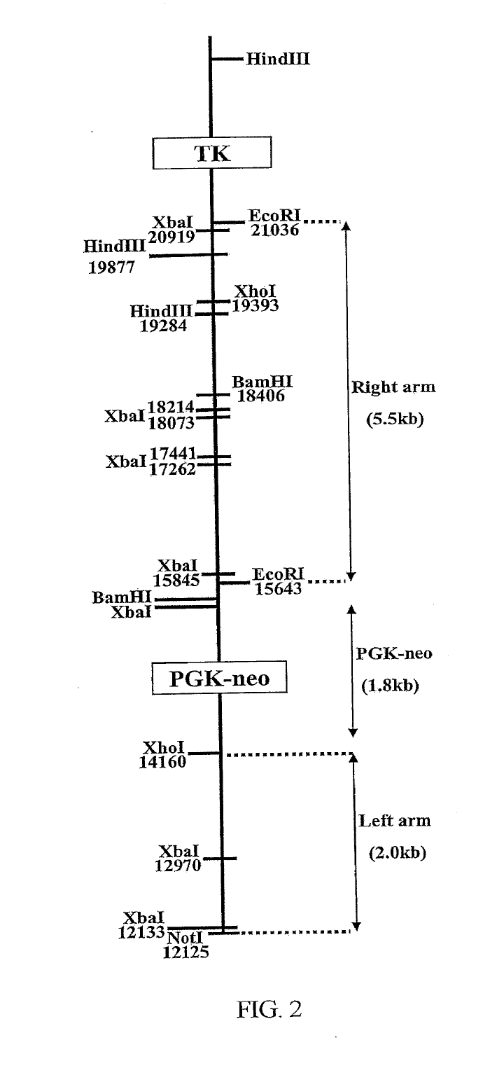

FIG. 3 shows the construction of a targeting vector where mutated human iduronate-2-sulfatase cDNA (C84T) or (R88P) is inserted.

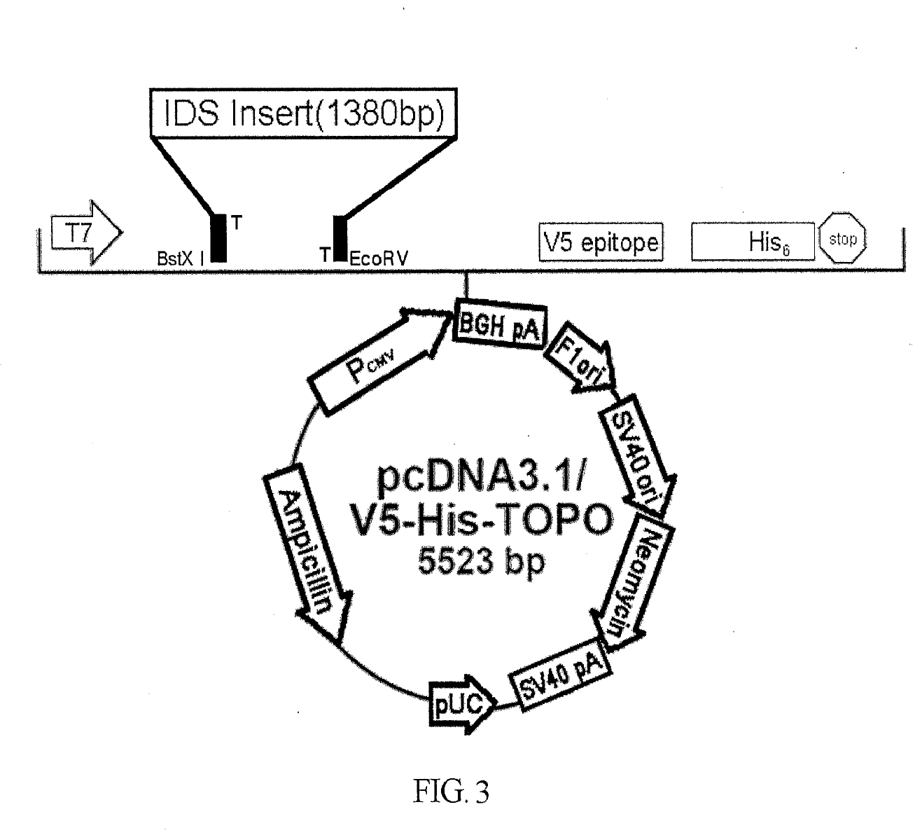

FIG. 4 shows the mutated human iduronate-2-sulfatase cDNA (C84T) or (R88P) construction in targeting vector.

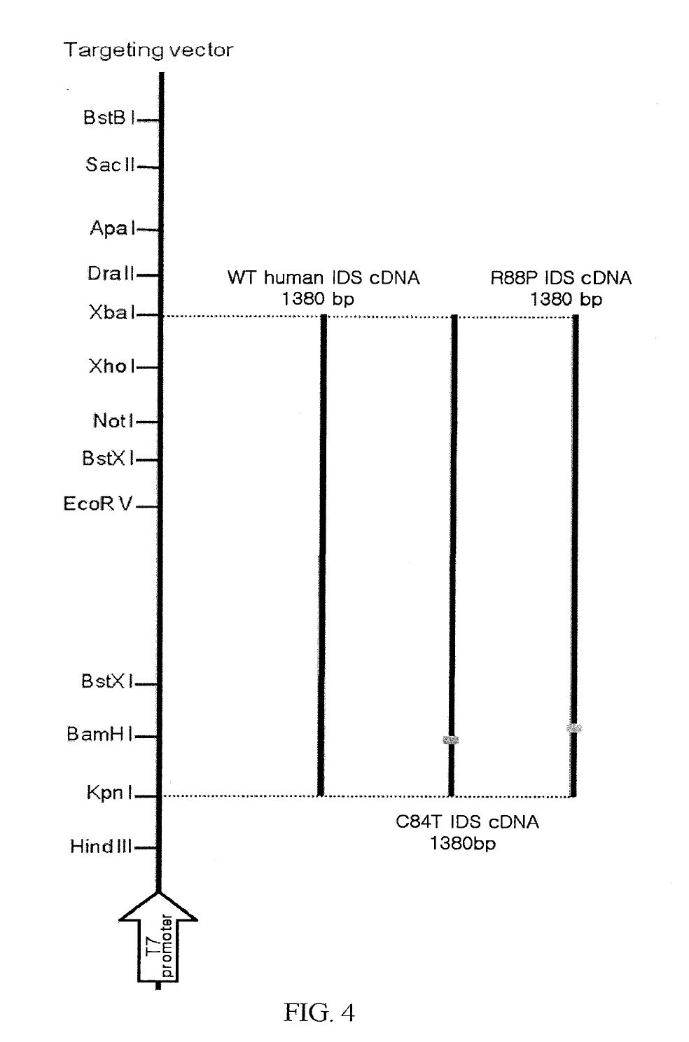

### DETAILED DESCRIPTION OF THE INVENTION

For preparing a transgenic mouse tolerant to human iduronate-2-sulfatase without generating anti-IDS IgG antibody, an iduronate-2-sulfatase knock-out mouse has to be prepared.

The first step for preparing a knock-out mouse is to construct a targeting vector. Said targeting vector consists of 2027 by of left arm, 1800 by of neomycin resistance gene (PGK-neo) and 5393 by of right arm. The left arm is constructed from 12133 residue to 14160 residue of wild type allele of iduronate-2-sulfatase gene. The right arm is constructed from 15643 residue to 21036 residue of wild type allele of iduronate-2-sulfatase gene. The neomycin resistance gene (PGK-neo) is flanked by LoxP sites where residue 14161 to residue 15642 of the iduronate-2-sulfatase gene are deleted.

Further, the deletion part of iduronate-2-sulfatase gene is located at exon 2 and exon 3 of human iduronate-2-sulfatase gene.

The PGK-neo construct is made by deleting exon 2 and exon 3 in iduronate-2-sulfatase gene. In its place, a neomycin resistance cassette is inserted. The mutated fragment is subcloned into the pGK-TK plasmid, which contains the HSV thymidine kinase (TK) gene driven by the PGK promoter in a Bluescript vector.

The second step for preparing a knock-out mouse is to electroporate the embryonic stem (ES) cells with linearized targeting vector, followed by plate on fibroblast feeder layers. ES cells are treated with geneticin (neomycin analog) to select for cells that had incorporated the targeting construct. ES cell clones surviving drug selection are screened for homologous recombination events by Southern blot analysis.

In the process of constructing the targeting vector, an exogenous XhoI restriction site and EcoRI restriction site are introduced at the 5′ end of the PGK-neo cassette and 3′ end of the PGK-neo cassette respectively. Therefore, ES cell genomic DNA is digested with XhoI and EcoRI and DNA blots are hybridized with a probe corresponding to an iduronate-2-sulfatase gene region located 5′ and 3′ to the integration site of the construct. With this strategy, the native allele is indicated by 9.2 Kb and a mutant allele produced by homologous recombination is indicated.

Finally, a single clone of ES cells that had undergone homologous recombination is microinjected into blastocysts and 10 chimeric mice are generated. Six of the chimeras demonstrate 90% chimerism by color. Five chimeric males transmit the mutated allele through the germline. Heterozygote offspring are identified by both PCR and Southern analysis of genomic DNA. Heterozygotes exhibit a grossly normal phenotype and normal fertility.

Genotyping 124 offspring from heterozygote crosses reveals the expected Mendelian ratios (+/+37/124, 29.8%; +/−54/124, 43.5% and −/−33/124, 26.6%) indicating no significant effect on embryo development.

On the other hand, the iduronate-2-sulfatase mutation mouse (C84T) or iduronate-2-sulfatase mutation mouse (R88P) also has to be prepared by the following process.

The first step for preparing the iduronate-2-sulfatase mutation mouse is to prepare mutated iduronate-2-sulfatase cDNA by 2 step PCR amplification to be used for construction of a targeting vector.

For preparing the iduronate-2-sulfatase mutation mouse (C84T), mutated cDNA in the DNA targeting construct has been prepared with the following steps: i) preparing human iduronate-2-sulfatase cDNA by amplifying wild type allele using forward primer of SEQ ID No:2 and reverse primer of SEQ ID No:3; and ii) preparing mutated human iduronate-2-sulfatase cDNA by amplifying the cDNA prepared in step (i) subjected to site directed mutagenesis using primer of SEQ ID No:4 and reverse primer of SEQ ID No:5.

For preparing the iduronate-2-sulfatase mutation mouse (R88P), mutated cDNA in the DNA targeting construct has been prepared with the following steps: i) preparing human iduronate-2-sulfatase cDNA by amplifying wild type allele using forward primer of SEQ ID No:2 and reverse primer of SEQ ID No:3; and ii) preparing mutated human iduronate-2-sulfatase cDNA by amplifying the cDNA prepared in step (i) subjected to site directed mutagenesis using primer of SEQ ID No:6 and reverse primer of SEQ ID No:7.

The second step for preparing the iduronate-2-sulfatase mutation mouse is to electroporate the embryonic stem (ES) cells with linearized targeting vector having mutated iduronate-2-sulfatase cDNA (C84T) or (R88P), followed by plate on fibroblast feeder layers. ES cells are treated with geneticin (neomycin analog) to select for cells that had incorporated the targeting construct. ES cell clones surviving drug selection are screened for homologous recombination events by Southern blot analysis.

Finally, a single clone of ES cells that had undergone homologous recombination is microinjected into blastocysts and 10 chimeric mice are generated. Six of the chimeras demonstrate 90% chimerism. Five chimeric males transmit the mutated allele through the germline. Heterozygote offspring are identified by both PCR and Southern analysis of genomic DNA. Heterozygotes exhibit a grossly normal phenotype and normal fertility. Genotyping 124 offspring from heterozygote crosses reveals the expected Mendelian ratios (+/+37/124, 29.8%; +/−54/124, 43.5% and −/−33/124, 26.6%) indicating no significant effect on embryo development.

A transgenic mouse tolerant to human iduronate-2-sulfatase without generating anti-IDS IgG antibody has been finally prepared by cross-breeding the knock-out mouse prepared in the preceding steps and mutation mouse (C84T) or (R88P) prepared in the preceding steps.

The finally prepared transgenic mouse tolerant to human iduronate-2-sulfatase without generating anti-IDS IgG antibody can be classified by 2 types.

One is a transgenic mouse tolerant to human iduronate-2-sulfatase resulted from cross breeding the knock-out mouse and mutation mouse (C84T). The other is a transgenic mouse tolerant to human iduronate-2-sulfatase resulted from cross-breeding the knock-out mouse and mutation mouse (R88P).

Transgenic mouse tolerant to human iduronate-2-sulfatase has the following physiological characteristics.

This transgenic mouse can express a phenotype of inactive iduronate-2-sulfatase, which does not cause the formation of anti-IDS IgG antibody. However, this transgenic mouse shows similar pathophysiological characteristics of the iduronate-2-sulfatase knock-out mouse, because expressed inactive iduronate-2-sulfatase cannot degrade glycosaminoglycan (GAG). Further, neutralized antibody is not generated in the course of IDS enzyme replacement in said transgenic mouse, which may result in adverse sensitization to the injected IDS enzyme.

Therefore, this transgenic mouse can be used as an optimal animal model for developing enzyme replacement therapy for iduronate-2-sulfatase.

The other aspect of the present invention is to provide a transgenic mouse model for developing enzyme replacement therapy for iduronate-2-sulfatase deficiency syndrome, wherein the target enzyme for treating iduronate-2-sulfatase deficiency syndrome has been experimented and selected by the following steps: i) preparing transgenic mouse tolerant to human iduronate-2-sulfatase without generating anti-IDS IgG antibody; ii) administering a candidate enzyme to said transgenic mouse; iii) measuring the efficacy of the candidate enzyme and adverse effect caused by anti-IDS IgG antibody; and iv) selecting the optimal enzyme showing maximum efficacy and minimum adverse effect among candidate enzymes.

Through use of the subject transgenic mouse, one can identify ligands or substrates that bind to, modulate, antagonize or agonize iduronate-2-sulfatase. Screening to determine drugs that lack effect on this enzyme is also of interest. Areas of investigation are the development of treating Hunter syndrome. Of particular interest are screening assays for enzymes that have a low toxicity for the human body.

A wide variety of assays may be used for this purpose including determination of the localization of drugs after administration, labeled in vitro protein-protein binding assays, protein-DNA binding assays, electrophoretic mobility shift assays, immunoassays for protein binding, and the like. Depending on the particular assay, a whole transgenic mouse may be used.

The term “enzyme” as used herein describes any molecule including iduronate-2-sulfatase, its analogue or its equivalent with the capability of affecting the biological action of iduronate-2-sulfatase. Generally, a plurality of assay mixtures can be run in parallel with different agent concentrations to obtain a differential response to the various concentrations. Typically, one of these concentrations serves as a negative control, i.e., at zero concentration or below the level of detection.

Candidate enzymes encompass numerous chemical classes, though typically they are enzymes. Candidate enzymes comprise functional groups necessary for structural interaction with enzymes, particularly hydrogen bonding, and typically include at least an amine, carbonyl, hydroxyl or carboxyl group, preferably at least two of the functional chemical groups.

Candidate enzymes are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries.

The therapeutic enzymes may be administered in a variety of ways, orally, topically, parenterally, e.g., subcutaneously, intraperitoneally, by viral infection, intravascularly, etc. Inhaled treatments are of particular interest. Depending upon the manner of introduction, the compounds may be formulated in a variety of ways. The concentration of therapeutically-active compound in the formulation may vary from about 1.0-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### EXAMPLES

The following examples relate to pathophysiological characteristics of a transgenic mouse tolerant to human iduronate-2-sulfatase without generating anti-IDS IgG antibody compared to that of a wild type mouse.

### Example 1

### Urine Amount of GAG

The urine amount of glycosaminoglycan (GAG) in a 38 weeks old transgenic mouse was in the range of 135-175 .μg/ml. On the other hand, the urine amount of GAG in a 38 weeks old wild type mouse was in the range of 10-30 .μg/ml. Therefore, the urine amount of GAG of the 38 weeks old transgenic mouse was 5-20 fold of that of the 38 weeks old wild type mouse. It means that the 38 weeks old transgenic mouse could not degenerate the GAG in the body, because the 38 weeks old transgenic mouse did not produce active iduronate-2-sulfatase in the body.

In case of a 16 weeks old transgenic mouse, the urine amount of GAG of the 16 weeks old transgenic mouse was in the range of 115-140 .μg/ml, while the urine amount of GAG of the 16 weeks old wild type mouse was in the range of 15-40 .μg/ml.

### Example 2

### Weight of Organ in the Knock-Out Mouse

The growth of liver was extremely retarded in the transgenic mouse. Further, the growth of spleens and lungs in the transgenic mouse was also retarded compared to those of the wild type mouse.

### Example 3

### Histological Analysis of Liver and Kidney in the Knock-Out Mouse

In the liver and kidney of the transgenic mouse, a lot of glycosaminoglycan (GAG) was accumulated in the lysosomes of these organs. Further, the growth and development of these organs in the transgenic mouse were shown to be retarded.

In case of the liver, the following pathological characteristics were shown. Lysosome-laden Kupffer cells are readily found at 4 weeks of age with very little evidence of significant hepatocyte storage. By 10 weeks of age, further progression of storage within the reticuloendothelial system has occurred and there is now evidence of significant hepatocyte vacuolation. At this age 20 to 30% of the cytoplasm of the hepatocytes appear to be taken up by lysosomes, as contrasted to very few discernible lysosomes within normal liver samples.

### Example 4

### Generation of Anti-IDS IgG Antibody

Elaprase® was administered to the transgenic mouse of the present invention for more than 24 weeks. However, any significant increase of anti-IDS IgG antibody could not be detected. On the other hand, the knock-out mouse prepared in the preceding steps showed an increase of anti-IDS IgG antibody.

What is claimed is:

1. A transgenic mouse tolerant to human iduronate-2-sulfatase without generating anti-IDS IgG antibody prepared by the steps comprising:
i) preparing an iduronate-2-sulfatase knock-out mouse comprising disruption in exon 2 and exon 3 in the iduronate-2-sulfatase gene, wherein said disruption has been introduced into its genome by homologous recombination with DNA targeting construct in an embryonic stem cell such that the targeting construct is stably integrated in the genome of said mouse, wherein the disruption of the iduronate-2-sulfatase gene results in an inability of said mouse to produce detectable levels of iduronate-2-sulfatase;
ii) preparing an iduronate-2-sulfatase mutation mouse comprising site direct mutagenesis of iduronate-2-sulfatase gene, wherein cysteine, the 84.sup.th amino acid of human iduronate-2-sulfatase of SEQ ID No:1 is replaced by threonine (C84T) or arginine, the 88.sup.th amino acid of human iduronate-2-sulfatase of SEQ ID No:1 is replaced by proline (R88P), wherein said mutation has been introduced into its genome with DNA targeting construct in an embryonic stem cell such that the targeting construct is stably integrated in the genome of said mouse, wherein the mutation of the iduronate-2-sulfatase gene results in capability of said mouse to produce inactive type of iduronate-2-sulfatase; and
iii) cross-breeding the knock-out mouse prepared in step (i)and the mutation mouse prepared in step (ii) for obtaining the transgenic mouse tolerant to iduronate-2-sulfatase.

2. The transgenic mouse tolerant to human iduronate-2-sulfatase according to claim 1, wherein said DNA targeting construct comprises left arm of 2027 bp, neomycin resistance gene (PGK-neo) of 1800 by and right arm of 5393 bp, wherein the left arm is constructed from residue 12133 to residue 14160 of wild type allele of human iduronate-2-sulfatase gene, the right arm is constructed from residue 15643 to residue 21036 of wild type allele of human iduronate-2-sulfatase gene, and said neomycin resistance gene (PGK-neo) is flanked by LoxP sites where residue 14161 to residue 15642 of wild type allele of human iduronate-2-sulfatase gene are deleted.

3. The transgenic mouse tolerant to human iduronate-2-sulfatase according to claim 1, wherein for iduronate-2-sulfatase mutation mouse (C84T), mutated cDNA in the DNA targeting construct has been prepared with following steps:
i) preparing a human iduronate-2-sulfatase cDNA by amplifying wild type allele using forward primer of SEQ ID No:2 and reverse primer of SEQ ID No:3; and
ii) preparing a mutated human iduronate-2-sulfatase cDNA by amplifying the cDNA prepared in step (i) subjected to site directed mutagenesis using primer of SEQ ID No:4 and reverse primer of SEQ ID No:5.

4. The transgenic mouse tolerant to human iduronate-2-sulfatase according to claim 1, wherein for iduronate-2-sulfatase mutation mouse (R88P), mutated cDNA in the DNA targeting construct has been prepared with following steps:
i) preparing human iduronate-2-sulfatase cDNA by amplifying wild type allele using forward primer of SEQ ID No:2 and reverse primer of SEQ ID No:3; and
ii) preparing mutated human iduronate-2-sulfatase cDNA by amplifying the cDNA prepared in step (i) subjected to site directed mutagenesis using primer of SEQ ID No:6 and reverse primer of SEQ ID No:7.

5. A transgenic mouse model for developing enzyme replacement therapy for iduronate-2-sulfatase deficiency syndrome,
wherein the target enzyme for treating iduronate-2-sulfatase deficiency syndrome has been experimented and selected by following steps:
i) preparing a transgenic mouse tolerant to human iduronate-2-sulfatase without generating anti-IDS IgG antibody;
ii) administering a candidate enzyme to said transgenic mouse;
iii) measuring the efficacy of candidate enzyme and adverse effect caused by anti-IDS IgG antibody; and
iv) selecting the optimal enzyme showing maximum efficacy and minimum adverse effect among candidate enzymes.
